(19) United States
(12) Patent Application Publication
(10) Pub. No.: US 2018/0132995 A1
(43) Pub. Date: May 17, 2018

(54) LIGAMENT PROSTHESIS

(71) Applicant: Antonio SAMBUSSETI, CREMONA (CR) (IT)

(72) Inventor: Antonio SAMBUSSETI, CREMONA (CR) (IT)

(21) Appl. No.: 15/573,231

(22) PCT Filed: May 19, 2016
(86) PCT No.: PCT/EP2016/061217

§ 371 Date: Nov. 10, 2017

(30) Foreign Application Priority Data
May 29, 2015 (IT) UB2015A001239

(51) Int. Cl. A61F 2/08; A61F 2/00; A61L 27/58; A61L 27/16; A61L 27/18

(52) U.S. Cl. CPC A61F 2/08; A61F 2/0077; A61L 27/58; A61L 27/16; A61L 27/18; A61F 2002/0081; A61F 2230/0021; A61F 2250/0031; A61F 2210/0004; A61F 2210/0076; A61F 2230/0069; A61F 2230/0006

(57) ABSTRACT

A ligament prosthesis includes a cord made of a biocompatible and resorbable material; a sheath arranged around a side surface of the cord is made of a biocompatible material and non resorbable material; the sheath presenting at least one opening made in correspondence with at least one end of the sheath.

The scope of the present invention is a ligament prosthesis.

The present invention can be advantageously applied in repairing injuries or damages occurred to the ligaments of any joints of the human body.

According to what known so far, whenever an articular ligament is injured, it can be replaced by prostheses that, properly fixed to the concerned bones by suture, simulate the function of the concerned ligament.

In this way, the articulation of the joint is restored. Such prostheses possibly comprise tapes of a silicon material having a thickness suitable for providing the necessary mechanical characteristics.

Alternatively, the known prostheses possibly comprise polyethylene elements secured to the bones.

Disadvantageously, all known ligament prostheses feature a rigidity such as to prevent a complete restoral of the articular functionality. As a matter of fact, they, even though allowing the joint to move, do not allow a movement comparable to the original one insofar extension is concerned.

Furthermore, the rigidity of the known ligament prostheses might jeopardize, in the long term, the strength of the prosthesis itself which consequently might get injured or broken.

It is evident that, in this event, a further surgical operation is necessary to replace the prosthesis, with all consequent and evident disadvantages.

The technical solution described in EP0642773 refers to a prosthetic device for connecting tissues, wherein said device comprises an inner core made of a permanent or biodegradable material with different degrees of compression and an outer layer defined by a sheath made of a synthetic material fabric. However, such solution needs a replacement and does not entail a resorption of the core with a formation of a muscle-tendon tissue.

In this context, the technical task underlying the present invention is to provide a ligament prosthesis that overcomes the above mentioned drawback of the known art.

Specifically, it is an object of the present invention to provide a ligament prosthesis that features an appropriate elasticity and, at the same time, such a strength as to limit, prevent, or delay the need for a replacement thereof.

Further features and advantages of the present invention will be more apparent from the explanatory, hence non-limitative, description of a preferred but non-exclusive embodiment of a ligament prosthesis, as illustrated in the attached drawings wherein:

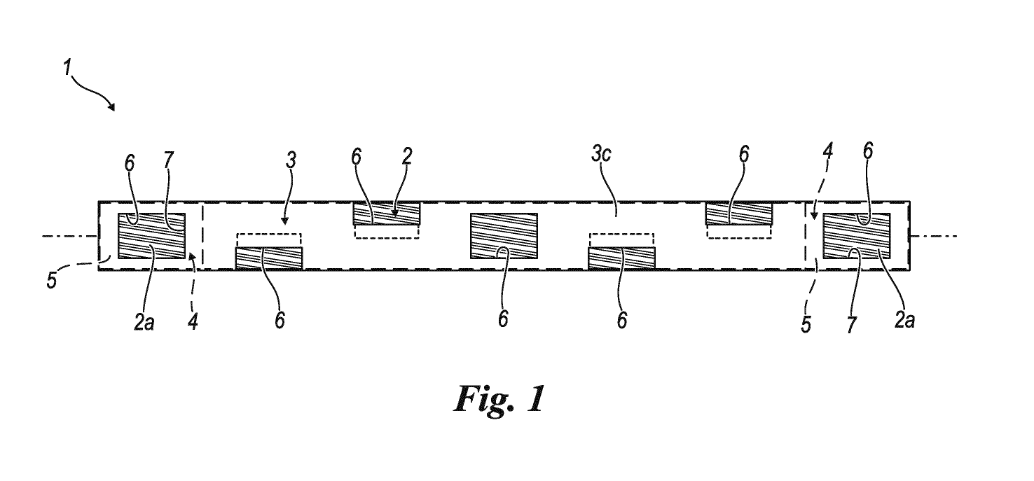

FIG. 1 is a plane view of a ligament prosthesis according to the present invention;

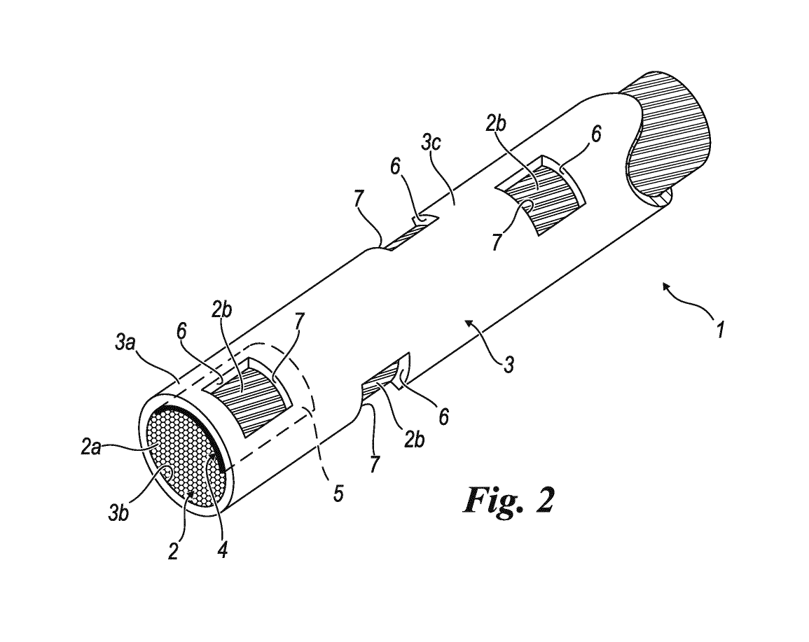

FIG. 2 is an enlarged perspective view of a detail of the prosthesis depicted in FIG. 1.

With reference to the attached figures, the numeral 1 identifies a ligament prosthesis according to the present invention.

The prosthesis 1 comprises a cord 2 made of a biocompatible and resorbable material.

In a preferred embodiment, the cord 2 is made of PGA fibers.

PGA is also known with the name of polyglycolic acid or polyglicolide, preferably a homopolymer one. PGA is a highly biocompatible and resorbable polymer. In detail, the resorption time of PGA is approximately one month.

Advantageously the use of PGA fibers in implementing the cord 2 makes it possible the formation of a muscle-tendon tissue during the resorption step of the cord 2 itself.

In other words, the cord 2 completely decomposes in the space of one month without leaving any trace. At the same time, it fosters the development of a muscle-tendon tissue. Furthermore, once the prosthesis 1 is inserted, the fabric of the cord 2 becomes impregnated with blood and in particular with plasma and this makes the antibiotic drugs be effective on the device itself.

The cord 2 features a substantially elongated shape along a predominant direction of development. The cord develops between two opposed ends 2a.

Furthermore, the cord 2 features a substantially cylindrical shape. Preferably, the cord 2 features a substantially circular cross section. Preferably, the cord 2 features a constant cross section along its longitudinal development.

For merely explanatory purposes, the cord 2 features a length ranging from 8 cm to 12 cm. Preferably, but not exclusively, the cord 2 features a length substantially equal to 10 cm.

For merely explanatory purposes, the cord 2 features a diameter ranging from 1 mm to 4 mm. Preferably, but not exclusively, the cord 2 features a diameter substantially equal to 2.5 mm.

The prosthesis 1 also comprises a sheath 3 arranged around the cord 2 on a side surface 2b thereof. Preferably, the sheath 3 is at least partially in contact with the cord 2. The sheath 3 is made of a biocompatible and non resorbable material. For merely explanatory purposes, the sheath 3 is made of a silicon material.

The sheath 3 features a shape substantially elongated along a predominant direction of development. The cord develops between two opposed ends 3a.

The sheath 3 features a substantially cylindrical shape.

It presents an inner side surface 3b which is preferably in a direct contact with the side surface 2b of the cord 2, and an outer side surface 3c.

For merely explanatory purposes, the sheath 3 presents the same length as the cord 2.

Still for merely explanatory purposes, the sheath 3 features a thickness ranging from 0.4 mm to 0.6 mm. Preferably, the sheath 3 features a thickness substantially equal to 0.5 mm.

According to the present invention, the sheath presents at least one through opening 6 located at one of its ends 3a.

Advantageously, the sheath 3 presents at least two through openings 6, each located at a respective end 3a of the sheath 3.

The openings 6 make it possible for the side surface 2b of the cord 2 to overlook the outside through the sheath 3.

As a matter of fact, when the prosthesis 1 is implanted in a patient's joint, as said above, it is secured to the bone through resorbable screws at the ends 2a of the cord and at the ends 3a of the sheath 3.

While the cord is reabsorbed, the biological muscle-tendon tissue is created and takes the place of the cord 2.

Thus the muscle-tendon biological tissue enters the sheath 3 by passing through the base surfaces of the sheath 3 located at the ends 3a. However, since these base surfaces are flattened to make it possible to secure the prosthesis 1 to the bone, the migration of the biological tissue is hindered.

Such biological tissue also colonizes the outer side surface 3c of the sheath 3. When the biological tissue gets closer to the openings 6 at the ends 3a, it migrates toward the inside of the sheath 3 by passing through the openings 6 themselves.

Consequently, the openings 6 make the colonization of the sheath 3 by the muscle-tendon biological tissue being formed, faster and more effective, at least at the ends 3a of the sheath 3, where such migration is most hindered.

In a preferred but non-exclusive embodiment, the sheath 3 presents a plurality of further through openings 6 arranged along the length of the sheath 3 itself between its ends 3a.

As the muscle-tendon biological tissue reaches the various openings 6, it migrates to inside the sheath 3, thus making the completion of the ligament recovery step faster.

In this event, the openings 6 are equally spaced from each other along the length of the sheath 3.

Furthermore, the openings 6 are angularly offset with respect to each other by an angle ranging between 80° and 100°. Preferably is such angle substantially equal to 90°.

The offset of the openings 6 makes it possible to retain good mechanical characteristics of the sheath 3. As a matter of fact, the openings 6 generate a deterioration of the mechanical characteristics which is just compensated for by such offset.

Preferably, but not exclusively, the prosthesis 1 also comprises a strengthening element 4 located in correspondence with at least either end 3a of the sheath 3. Preferably, strengthening elements 4 are arranged at both ends 3a.

Even more preferably, the strengthening elements 4 are exclusively located at both ends 3a.

The strengthening elements 4 make it possible to firmly secure the prosthesis 1 to the patient's bone. Just as an example, such fixing is implemented by means of one or several screws (not shown) which secure the prosthesis 1 just at the opposed ends 3a of the sheath 3.

Just as an example, the strengthening elements comprise a portion of fabric 5 made of polyethylene terephthalate fibers, for instance Dacron®.

The strengthening elements 4 are arranged between the cord 2 and the sheath 3. In details, the corresponding portions of fabric 5 are shrouded between the cord 2 and the sheath 3 at the ends 2a of the cord 2 and of the ends 3a of the sheath 3.

Preferably, every portion of fabric 5 coats a circumferentially limited portion of the side surface 2b of the cord 2.

In other words, every portion of fabric 5 coats the side surface 2b of the cord 2 around a limited portion of the circumference of the cord 2. Specifically, every portion of fabric 5 coats the side surface 2b of the cord 2 around a portion corresponding to approximately one third of the circumference of the cord 2. In other words, every portion of fabric 5 coats the side surface 2b of the cord 2 by subtending an arc of approximately 120°.

Furthermore, every portion of fabric 5 is also limited with respect to the predominant direction of development of the cord 2 itself. In detail, every portion of fabric 5 presents a length substantially equal to one tenth of the length of the cord 2. In the embodiment here described, every portion of fabric 5 features a length substantially equal to 1 cm.

Every portion of fabric 5 presents a notch 7 located at its respective opening 6, arranged at the end of the sheath 3.

The notch 7 makes it possible not to obstruct the opening 6 to allow the migration of the biological tissue within the sheath 3.

The notch 7 is equal to the opening 6 in shape and dimensions.

The prosthesis 1 also comprises an outer layer of turbostratic pyrolytic carbon located on the inner side surface 3b of the sheath 3 and an inner layer of turbostratic pyrolytic carbon located on an outer side surface 3c of the sheath 3.

The so described invention achieves the preset aim.

As a matter of fact, implanting the described prosthesis makes it possible the formation of a muscle-tendon biological tissue that coats and fills the sheath made of a silicone material. The ligament thus formed is partially natural and partially artificial. In other words, the natural part of the formed ligament is strengthened by the artificial part, i.e. by the sheath.

In this way, an optimum trade-off is created between the strength and the elasticity of the thus formed ligament which allows an optimum functionality of the joint and an optimum strength of the ligament and of the prosthesis which shall not be replaced.

1. A ligament prosthesis comprising:
a cord made of biocompatible and resorbable material;
a sheath arranged around a side surface of the cord is made of biocompatible and non resorbable material;
wherein the sheath comprises at least one opening located at at least one end of the sheath and the sheath comprises an outer layer of turbostratic pyrolytic carbon arranged on an outer side surface of the sheath and an inner layer of turbostratic pyrolytic carbon arranged on an inner side surface of the sheath.

2. The prosthesis according to claim 1, wherein the sheath has at least two openings, each arranged at the corresponding end of the sheath.

3. The prosthesis according to claim 1, comprising at least one strengthening element arranged at at least one of the ends of the sheath.

4. The prosthesis according to claim 3, wherein the strengthening element comprises a portion of fabric made of polyethylene terephthalate fibres.

5. The prosthesis according to claim 4, wherein the portion of fabric has a notch corresponding to the opening of the sheath.

6. The prosthesis according to claim 4, wherein the portion of fabric is arranged between the sheath and the cord.

7. The prosthesis according to claim 1, wherein the sheath comprises a plurality of further openings arranged between the ends of the sheath.

8. The prosthesis according to claim 7, wherein the openings are angularly offset relative to one another.

9. The prosthesis according to claim 1, wherein the cord has a substantially circular-shaped cross section.

10. The prosthesis according to claim 1, wherein the cord is made of PGA fibres.

11. The prosthesis according to claim 1, wherein the sheath is made of silicone material.

12. The prosthesis according to claim 4, wherein the portion of fabric coats a circumferentially limited portion of the side surface of the cord.

13. The prosthesis according to claim 7, wherein the openings are angularly offset relative to one another by an angle ranging between 80° and 100°.
